(19) United States
(12) Patent Application Publication
(10) Pub. No.: US 2026/0007338 A1
(43) Pub. Date: Jan. 8, 2026

(54) DERMAL SENSOR INSERTION AND RETENTION

(71) Applicant: Laxmi Therapeutic Devices, Inc., Goleta, CA (US)

(72) Inventors: Tom Metzmaker, Goleta, CA (US); Matthew Yavorsky, Granada Hills, CA (US); William Peter Van Antwerp, Santa Clarita, CA (US); Jason Zerweck, Media, PA (US)

(21) Appl. No.: 19/258,180

(22) Filed: Jul. 2, 2025

Related U.S. Application Data
(60) Provisional application No. 63/667,427, filed on Jul. 3, 2024.

(51) Int. Cl. A61B 5/145; A61B 5/00

(52) U.S. Cl. CPC A61B 5/14546; A61B 5/6848

(57) ABSTRACT

An applicator for applying an analyte monitor to a patient's skin includes a surface configured to hold at least a base of the analyte monitor, a delivery needle configured to advance axially past the surface and the base when the base is held to the applicator to deliver a portion of a sensor member of the analyte monitor into the patient's skin at an insertion site located within a perimeter of the base, and an actuator configured to be actuated to initiate advancement of the delivery needle. When the surface of the applicator is held against the patient's skin, at least part of the actuator is positioned directly above the insertion site of the delivery needle, and the actuator is configured to be actuated in a direction perpendicular to the surface of the patient's skin.

### CROSS-REFERENCE TO RELATED APPLICATION(S)

The present application claims priority to and the benefit of U.S. Provisional Patent Application No. 63/667,427, filed Jul. 3, 2024, the contents of which are hereby incorporated by reference in their entirety.

### BACKGROUND

### Field

The present disclosure relates to improvements in sensor insertion and retention mechanisms for analyte sensors, methods for improving insertion and retention of such analyte sensors, and instruments to facilitate such improvements. In some cases, the analyte sensors may measure analytes, such as glucose, in a body of a subject.

### Description of Related Art

Monitoring different analytes in the human body can be used for various diagnostic reasons. In particular, monitoring glucose levels is important for individuals suffering from type 1 or type 2 diabetes. People with type 1 diabetes are unable to produce insulin or produce very little insulin, while people with type 2 diabetes are resistant to the effects of insulin. Insulin is a hormone produced by the pancreas that helps regulate the flow of blood glucose from the bloodstream into the cells in the body where it can be used as a fuel. Without insulin, blood glucose can build up in the blood and lead to various symptoms and complications, including fatigue, frequent infections, cardiovascular disease, nerve damage, kidney damage, eye damage, and other issues. Individuals with type 1 or type 2 diabetes need to monitor their glucose levels in order to avoid these symptoms and complications.

Analyte monitors, and in particular, glucose monitors for the monitoring of glucose levels for the management of diabetes, are constantly being developed and improved. Although there are several platforms for monitoring analytes such as glucose available on the market, there is still a need to improve their precision, wearability, and accessibility to end-users. In addition, there is a desire to provide robust, less painful, less error-prone, and/or generally more effective continuous glucose monitors which may be attached to the patient's body for a more prolonged period of time, as well as glucose monitor features that can be used together with such improved monitor and monitor designs. Furthermore, there is also a desire to provide improved applicators and applicator designs for proper and consistent application of such improved monitors.

In particular, as continuous glucose monitors improve, for example, as life cycles of monitors have proposed to increase in certain applications and as the size of sensor members of monitors generally decreases (i.e., the portions of sensor members intended to be implanted under a patient's skin are being pushed to be made smaller and less invasive, for example, to reduce discomfort to the patient), it has become more difficult both to provide proper and accurate insertion of the smaller sensors, as well as to retain said smaller sensor members at a proper position/orientation and depth under the patient's skin for the longer durations of use, sometimes on the order of multiple weeks.

### SUMMARY

Many continuous glucose monitors are intended to be worn on a patient's skin for a duration of multiple days or weeks. Most or all commercially available glucose sensors on the market today sense glucose in interstitial fluid (ISF) below the surface of the skin. Such sensing or monitoring therefore typically involves an initial step of inserting a sensor of the glucose monitor under the patient's skin. For the most part, this insertion step will involve puncturing the surface of the skin, for example, with a separate needle, for example, on an applicator to provide access for inserting the sensor. Thereafter, the needle or other sharp may be retracted, while the sensor member stays in place under the patient's skin. Interaction between an applicator for delivering the needle and sensor combination and the patient is improved according to embodiments of the invention, so that a more consistent and accurate implantation of the sensor member under the patient's skin can be achieved.

In addition, continuous glucose monitors may include a device body that remains adhered to the patient for a prolonged period of time as well, where the device body is connected to and communicates with the portion of the sensor member under the patient's skin to gather information about the patient's glucose levels or other analyte levels. According to embodiments of the invention, adhesive patches associated with these device bodies may also be leveraged and improved to improve accurate and consistent delivery of the sensor member under the patient's skin. In other embodiments, a shape of the sensor may also be designed to provide improved retention and positioning of the sensor member under the patient's skin.

Embodiments of the invention are directed both to delivery methods and mechanisms associated with more accurately and consistently implanting a sensor member under a patient's skin, as well as methods and mechanisms for more effectively retaining and holding a desired position of a sensor member under the patient's skin in order to provide more accurate analyte readings for the patient or end user. The improved delivery methods according to embodiments of the invention are directed both to the designs of the analyte monitors being adhered to and/or implanted in the patient, as well as applicators that can more effectively and accurately implant the associated sensor members of such analyte monitors under the skin of patients.

According to an embodiment of the invention, an applicator for applying an analyte monitor to a patient's skin includes a surface configured to hold at least a base of the analyte monitor for positioning the base against the patient's skin and adhering the base to the patient's skin, a delivery needle configured to advance axially past the surface and the base when the base is held to the applicator to deliver a portion of a sensor member of the analyte monitor into the patient's skin at an insertion site located laterally within a perimeter of the base, and an actuator configured to be actuated to initiate advancement of the delivery needle to the insertion site and into the patient's skin. When the surface of the applicator is held against the patient's skin to adhere the base to the patient's skin, at least part of the actuator is positioned directly above the insertion site of the delivery needle relative to an axis that is normal to a surface of the patient's skin, and the actuator is configured to be actuated in a direction parallel to the axis that is normal to the surface of the patient's skin.

The surface of the applicator may further be configured to hold at least a transmitter of the analyte monitor.

The delivery needle may be configured to advance linearly past the surface and the base when the actuator is actuated. The delivery needle may further be configured to advance linearly at an acute angle relative to the surface of the patient's skin.

The delivery needle may be configured to advance along a curved trajectory past the surface and the base when the actuator is actuated.

After the portion of the sensor member is delivered into the patient's skin at the insertion site, the delivery needle may be configured to be retracted back above the surface and the base and into the applicator while the portion of the sensor member remains in the patient's skin.

The actuator may include a button or trigger.

A minimum force needed to actuate the actuator may correspond to a desired force to be applied by the actuator to the patient's skin at the insertion site to constrain a region of the patient's skin around the insertion site.

According to another embodiment of the invention, an applicator for applying an analyte monitor to a patient's skin includes a surface configured to hold at least a base of the analyte monitor for positioning the base against the patient's skin and adhering the base to the patient's skin, a delivery needle configured to advance axially past the surface and the base when the base is held to the applicator to deliver a portion of a sensor member of the analyte monitor into the patient's skin at an insertion site located laterally within a perimeter of the base, and an actuator configured to be actuated to initiate advancement of the delivery needle to the insertion site and into the patient's skin. When the surface of the applicator is held against the patient's skin to adhere the base to the patient's skin, the delivery needle is configured to be advanced along an axis that is arranged at an acute angle relative to a surface of the patient's skin to deliver the sensor member under the surface of the patient's skin at the acute angle, while the actuator is configured to be actuated in a direction perpendicular to the surface of the patient's skin, such that respective axes of movement of the delivery needle and the actuator are angled relative to one another.

The surface of the applicator may further be configured to hold at least a transmitter of the analyte monitor.

After the portion of the sensor member is delivered into the patient's skin at the insertion site, the delivery needle may be configured to be retracted back above the surface and the base and into the applicator while the portion of the sensor member remains in the patient's skin.

The actuator may include a button or trigger.

The acute angle may be between 20° and 40°. More specifically, the acute angle may be approximately 30°.

According to yet another embodiment of the invention, a system includes an analyte monitor and an applicator for applying the analyte monitor to a patient's skin. The applicator includes a surface configured to hold at least a base of the analyte monitor for positioning the base against the patient's skin, a delivery needle configured to advance axially past the surface and the base when the base is held to the applicator to deliver a portion of a sensor member of the analyte monitor into the patient's skin at an insertion site located laterally within a perimeter of the base, and an actuator configured to be actuated to initiate advancement of the delivery needle to the insertion site and into the patient's skin. When the surface of the applicator is held against the patient's skin, an adhesive patch is configured to adhere the base to the patient's skin, the adhesive patch further configured to constrain a region of the patient's skin surrounding the insertion site, and wherein actuation of the actuator is configured to apply direct pressure against a portion of the adhesive patch that surrounds the insertion site and that is closer radially to the insertion site than to an outer perimeter of the base to increase tension of the patient's skin that surrounds the insertion site during advancement of the delivery needle.

The surface of the applicator may be configured to hold at least a transmitter of the analyte monitor.

After the portion of the sensor member is delivered into the patient's skin at the insertion site, the delivery needle may be configured to be retracted back above the surface and the base and into the applicator while the portion of the sensor member remains in the patient's skin.

The sensor member may be configured to be inserted to a maximum depth of 1.5 mm to 3 mm deep in the patient's skin. More specifically, the sensor member may be configured to be inserted to a maximum depth of less than 2.5 mm deep in the patient's skin, so as to position sensing regions of the sensor member in the dermis of the patient's skin.

When the surface of the applicator is held against the patient's skin, a distance between the insertion site and the portion of the adhesive patch that surrounds the insertion site where the direct pressure is applied by the actuator may be less than 1 cm.

### BRIEF DESCRIPTION OF THE DRAWINGS

Further features and advantages of the invention will become apparent from the description of embodiments by means of the accompanying drawings. In the drawings:

FIGS. 1A and 1B schematically show a human body with an analyte monitor including an analyte sensor according to embodiments of the invention, where the analyte monitor is attached at different positions on the body.

FIG. 2 shows a perspective view from above an exemplary analyte monitor including an analyte sensor according to embodiments of the invention.

FIG. 3 shows a perspective view from below the analyte monitor of FIG. 2.

FIG. 4A shows a cutaway view of a portion of a patient's skin with a schematic depiction of a typical sensor member of an analyte monitor implanted therein, while FIG. 4B shows a cutaway view of a portion of a patient's skin with a schematic depiction of a sensor member of an analyte monitor according to an embodiment of the invention, e.g., the analyte monitor shown in FIGS. 2-3, implanted therein.

FIG. 5 shows a cross-sectional view from a side of a portion of a patient's skin with a further schematic depiction of a sensor member of an analyte monitor according to an embodiment of the invention implanted therein.

FIG. 6 shows an enlarged perspective view of a portion of a sensor member of an analyte monitor according to an embodiment of the invention.

FIG. 7 shows a cross-sectional view from a side of an applicator according to an embodiment of the invention configured to implant a sensor member of an analyte monitor according to an embodiment of the invention.

FIG. 8 shows an enlarged perspective view of a portion of a delivery needle of the applicator of FIG. 7 holding a portion of a sensor member of an analyte monitor according to an embodiment of the invention to be implanted under a patient's skin.

### DETAILED DESCRIPTION

In the following detailed description, only certain embodiments of the subject matter of the present disclosure are described, by way of illustration. As those skilled in the art would recognize, the subject matter of the present disclosure may be embodied in many different forms and should not be construed as being limited to the embodiments set forth herein.

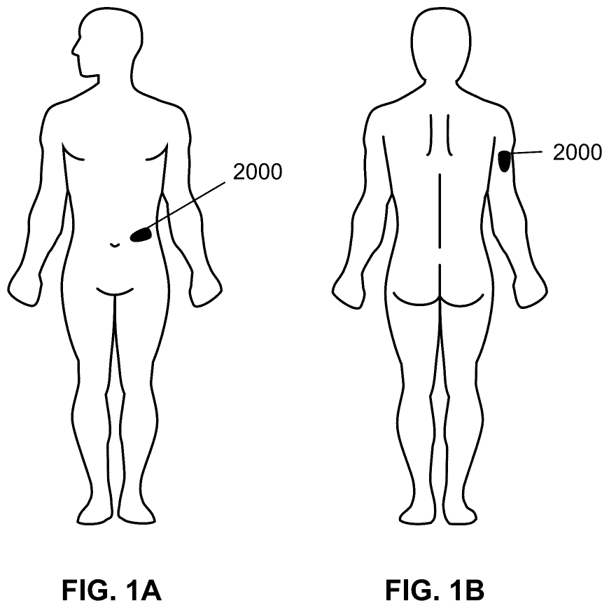

Monitors that include analyte sensors, such as glucose monitors, and in particular continuous glucose monitors, can be attached to a patient's body in different locations, in order to for example, improve glucose monitoring and/or a patient's comfort, since the continuous glucose monitors must remain adhered to the patient's skin, sometimes for a few days or more. FIG. 1A shows a first exemplary analyte monitor 2000 that is adhered to a patient's abdominal region, while FIG. 1B instead shows the exemplary analyte monitor 2000 adhered to a patient's arm. These are only meant to be example adhesion sites, and in other situations, this or a similar analyte monitor may instead be adhered or otherwise attached to other parts of the patient's body.

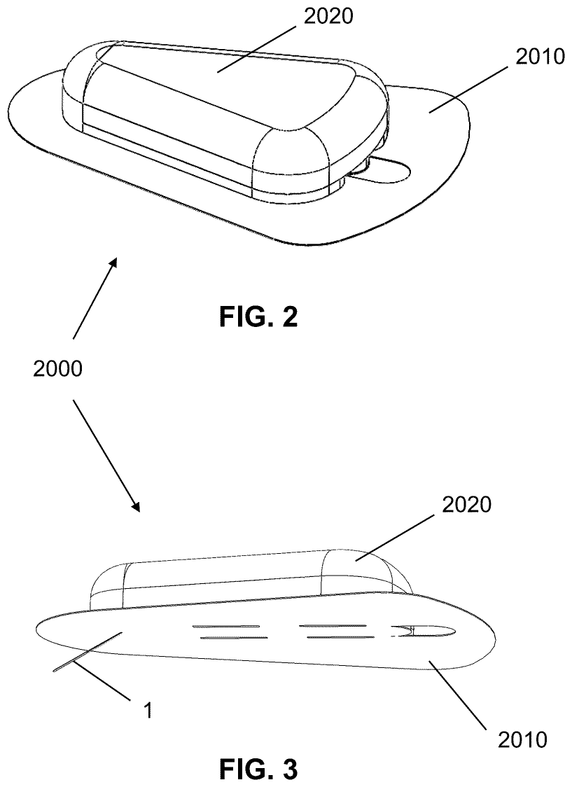

FIGS. 2 and 3 show different schematic views of an example analyte monitor 2000, which can be a continuous glucose monitor, according to an embodiment of the invention. The continuous glucose monitor 2000 may include a base or cradle 2010 that may have an adhesive layer for adhering to a patient's skin, a transmitter 2020 for transmitting data to and/or from a location away from the monitor, and a sensor member 1 which may include an integrated analyte sensing region such as a glucose sensor. It is to be understood that the example analyte monitor shown in FIGS. 2 and 3 are for illustrative and descriptive purposes only, and that analyte monitors with different structures and functionality can also be used in conjunction with the sensor assemblies described below, without departing from the spirit or scope of the invention.

The base 2010 will generally include an adhesive patch or other adhesive mechanism on its lower surface in order to facilitate attachment of the base 2010 to the surface of the patient's skin. In some embodiments of the invention, the sensor member 1 may be integrally formed with the base 2010 so as to be implanted under the patient's skin upon attachment of the base 2010 to the surface of the skin, while in other embodiments the base 2010 may be applied to the skin first, and the sensor member 1 may be advanced to a position where the sensor member 1 becomes attached to the base during or after implantation of part of the sensor member 1 under a patient's skin. For some embodiments, application of the base 2010 and/or the sensor member 1 may be facilitated with one or more applicators. An applicator according to an embodiment of the invention that can be used to apply both the base 2010 and the sensor member 1 to a patient will be described in greater detail below.

In some embodiments, after the base 2010 has been attached to a surface of the patient's skin and after at least part of the sensor member 1 has been advanced under the patient's skin, the transmitter 2020 which may be a separate part can be attached to the base. In some embodiments, the transmitter 2020 may include, for example, a power source such as a battery, while the base 2010 and/or the sensor member 1 may include additional electrical circuitry or contacts to complete a circuit, such that the monitor 2000 is powered up upon assembly of the transmitter 2020 to the base 2010. Other embodiments may include other arrangements, for example, where the battery is housed in the base 2010 instead of on the transmitter 2020, etc. Still other embodiments may include further different arrangements, for example, an integrally manufactured monitor where the base and transmitter are formed together in a single main body and which may not be separable from one another by the end user, where such embodiments may also include an integrated sensor member, or may include a sensor member that is implanted through the main body of the monitor after the main body has been adhered to the surface of the patient's skin.

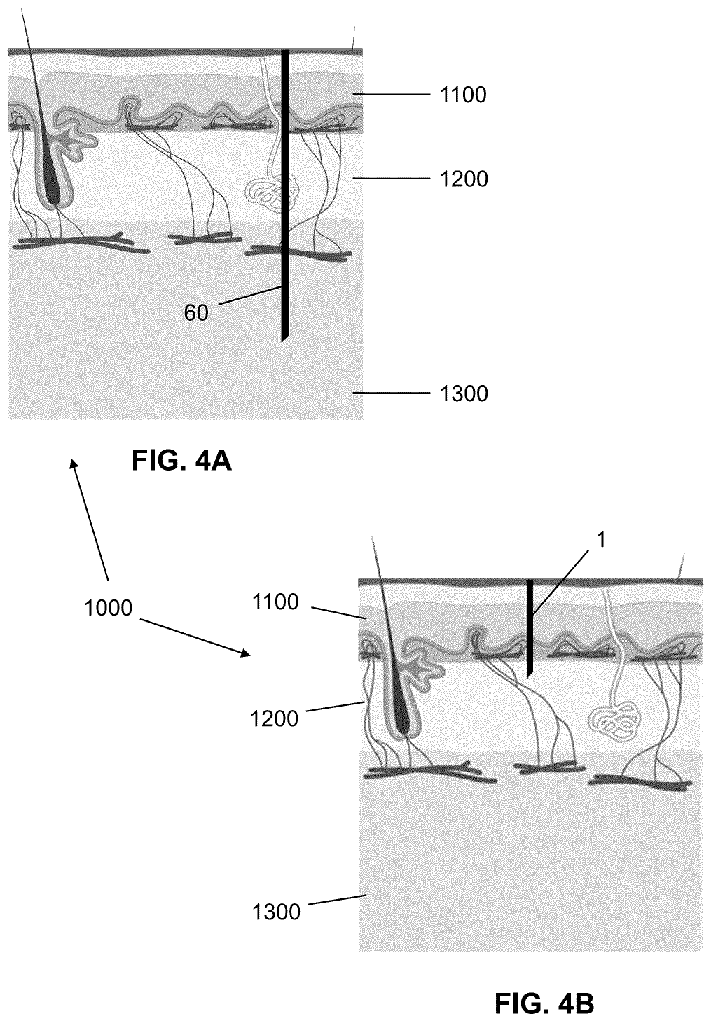

In addition to the above, embodiments of the invention may incorporate a sensor member 1 that does not extend as deeply into a patient's skin as more traditional monitor sensors. For example, as seen in FIGS. 4A and 4B, the skin 1000 of a human includes various layers, such as an epidermis 1100 that is closest to the surface of the skin, an intermediate dermis 1200, and a deeper hypodermis 1300. As shown schematically in FIG. 4A, sensor members 60 for typical commercially available monitors generally extend deeply into the skin, with sensing regions that must be positioned in the hypodermis 1300, approximately 6 mm to 7 mm below the surface of the skin. As such, most sensor members 60 need to both be long enough to extend into the hypodermis 1300, as well as sturdy enough to be held at that depth, thereby generally necessitating a thicker overall sensor member as well. Consequently, particularly in the case of continuous glucose monitors that may be worn for longer durations such as multiple days or weeks, these longer sensors may be more abrasive, and/or cause more trauma or discomfort to the patient, both during insertion or implantation, as well as during the life of the sensor. Furthermore, the hypodermis 1300 of the skin 1000 is generally more fatty than the epidermis 1100 and the dermis 1200, and so when the sensing region is positioned in the hypodermis 1300, the increased levels of fat in the hypodermis 1300 may delay glucose transport, and cause a time lag associated with a useable signal from the sensor member. In some cases, the time lag caused by the increased fat levels in the hypodermis 1300 compared to the dermis 1200 or epidermis 1100 before a useable signal can be obtained by the sensor member may be 15 minutes or more, which may cause a significant delay in data collection and analysis by the monitor, as well as potential inaccuracies if an inaccurate signal is used or incorporated into later data analyses.

In contrast, as shown schematically in FIG. 4B, sensor members 1 according to embodiments of the invention may only extend more shallowly into the shallow regions of the dermis 1200, with sensing regions in either the shallow regions of the dermis 1200, the epidermis 1100, or both, rather than requiring the sensing regions to extend more deeply into the hypodermis 1300. Positioning the sensing regions of the sensor member 1 in the shallow regions of the dermis 1200 or the epidermis 1100 may have many advantages. For example, the sensor member 1 according to embodiments of the invention can be made smaller, both in terms of length and thickness, which may reduce trauma during insertion and/or discomfort during the life of the sensor/monitor. The reduction in size of the sensor member 1 compared to traditional sensor members 60, or more generally, the reduced depth to which the sensor member 1 extends under the skin, will also allow sensing in less fatty layers of the skin, consequently resulting in faster glucose transport, more accurate signal readings by the sensor member, and a shorter delay or time lag associated with being able to retrieve useable signals from the sensor member 1. Generally speaking, sensor members 1 according to embodiments of the invention may extend to approximately ⅓ of the depth of traditional sensor members 60, for example, between 1.5 mm to 3 mm below the surface of the skin, but the sensor member in other embodiments may be positioned shallower or deeper compared with traditional sensor members, without departing from the spirit or scope of the invention, so long as the sensing regions are not intended to be positioned deeper than the shallow regions of the dermis 1200. Targeting the shallower regions of the dermis 1200 furthermore allows for some tolerance or leeway, where for example, if a sensor member 1 is implanted at a slightly more vertical angle than intended, the sensing regions of the sensor member 1 should still be positioned in the dermis 1200 rather than deeper in the hypodermis 1300, and still benefit from both reduced discomfort and less glucose transport delays due to fat in the tissue.

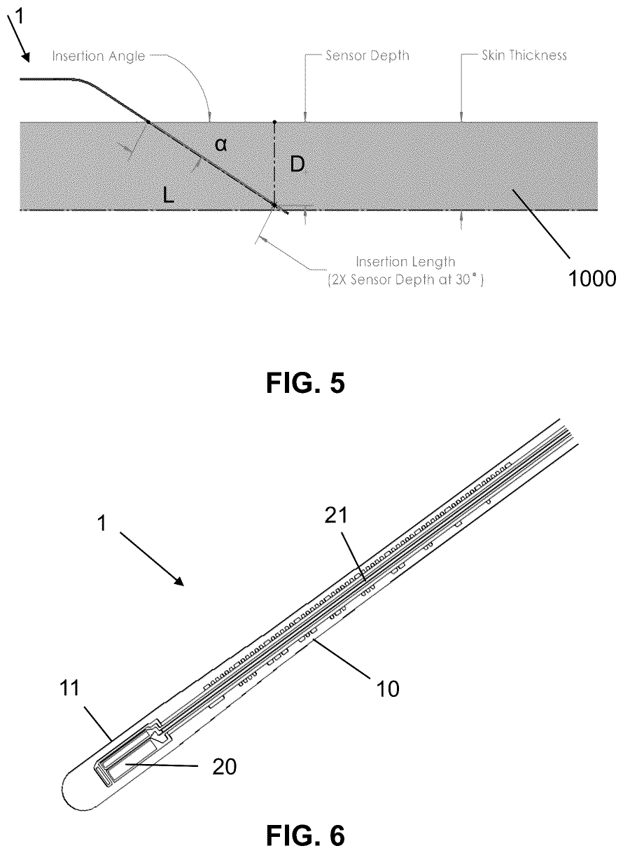

FIG. 5 provides a side cross-sectional view of another schematic sensor member 1 that is implanted in skin 1000 according to an embodiment of the invention. Similarly as previously discussed, while not depicted in FIG. 5, the sensor member 1 may extend into the skin 1000 to an approximate depth of the shallow dermis of the skin, such that the sensing regions located at or near a distal end of the sensor member 1 are positioned in the epidermis or shallow dermis for sensing. When the sensor member 1 is implanted at a shallower depth, retention issues that are not present for deeper extending sensor members may arise. Movements by the patient, for example, manipulation of the skin or underlying muscles, may cause the implanted portion of the sensor member 1 to move, and when the sensor member does not extend as deeply into the skin, such movements may cause the implanted portion of the sensor member 1 to partially withdraw, or may potentially even completely pull out of the skin. This issue may be further exacerbated in situations where the sensor member 1 is implanted into the skin at an angle substantially perpendicular to the surface of the skin, where in such cases, the length of the sensor member 1 implanted into the skin is the same as the depth of the sensor member, so that a movement that dislodges the sensor member 1 or its corresponding monitor by about that length/depth may potentially cause an unwanted withdrawal or pulling out of the sensor member 1 from the skin, which would disrupt the monitoring of the patient.

As such, as shown in FIG. 5, according to embodiments of the invention, the sensor member 1 can be implanted under the skin at an acute entry angle α relative to a surface plane of the skin 1000 instead. By implanting sensor member 1 at an angle α, the length of the sensor that is actually implanted into the skin can be increased. For example, as shown in FIG. 5, if the entry angle α is 30° relative to the surface of the skin 1000 and the sensor member 1 is implanted to a depth D, the actual length L of the portion of the sensor member 1 that is implanted will be two times the depth D. Just by way of example, if the entry angle α is 45° relative to the surface of the skin 1000, the actual length L of the portion of the sensor member 1 that is implanted will be about 1.4 times the depth D. Therefore, by varying the angle of implantation, a larger portion of the sensor member 1 can be implanted under the skin 1000 without the sensor member reaching an undesirably large depth, e.g., without the sensor member 1 entering the hypodermis. And by way of a larger length of sensor member 1 being implanted under the skin 1000, retention of the sensor member 1 under the skin 1000 can be improved, so that it will be more difficult to dislodge the sensor member 1 from withdrawing and pulling out of the skin 1000. Furthermore, an angled insertion according to embodiments of the invention will allow for more consistent placement of the sensor member 1 in the correct tissue space, e.g., the epidermis or the shallow dermis, and will provide more tolerance or leeway to do so.

Construction of the sensor member 1 according to an embodiment of the invention will now be discussed in greater detail, with reference to FIG. 6. As seen in FIG. 6, a sensor member 1 may include an implantable portion that includes a main body 10 and a sensing region including one or more sensing electrodes 20. The main body 10 may include a sensor tip or distal end region 11 that is enlarged in a width direction compared to other portions of the main body. For example, in one embodiment, the distal end region 11 may be substantially teardrop-shaped or otherwise widened with a tapering profile. In other embodiments, the distal end region may include arms or teeth extending laterally outwardly, or may include other shapes or profiles that provide for a varying width. Enlarging the distal end region 11 can help anchor the electrodes at a particular position under the surface of the skin and facilitate improved positioning and retention of the sensor member 1 under the skin after implantation. In one embodiment, the distal end region 11 may be approximately 38% wider than another portion of the main body 10, for example, if a portion of the main body 10 has a width of 300 μm, the distal end region 11 may have a width of 416 μm. Other ratios and sizes may also be contemplated without departing from the spirit or scope of the invention. For example, the distal end region 11 may be 20% to 150% wider than another portion of the main body 10, more preferably 25% to 75% wider than another portion of the main body, and still more preferably 30% to 50% wider than another portion of the main body. The sensing electrodes 20 may further be electrically connected to leads 21 that extend along the main body 10 of the sensor and to an opposite proximal end of the sensor member 1, to facilitate electrical connectivity and communication of the sensing electrodes 20 with other electrical components of the monitor.

According to an embodiment of the invention, the sensor member 1 may further be manufactured or otherwise formed to be more flexible than traditional sensors. Fabrication techniques may be utilized, for example, to fabricate the sensor member 1 according to embodiments of the invention on a thin film substrate or other similar flexible substrate, in order to promote increased flexibility of the sensor. Fabrication techniques can also be utilized to facilitate reducing the thickness of the sensor member 1 according to embodiments of the invention, to further increase flexibility. By way of example, typical stiffer sensor members may be around 200 μm in thickness, whereas sensor members 1 according to embodiments of the invention may be manufactured to be less than half as thick, for example, 75 μm. Other similar reductions in sizes may further be realized without departing from the spirit or scope of the invention.

The combination of having a widened distal end portion, a thinner and generally smaller and/or more flexible substrate, and/or adjusting for a more effective angle of insertion increases the overall flexibility of the sensor, allowing it to move together with the body more freely, thereby improving retention by both increasing the length of sensor member that has to be pulled out to avoid full withdrawal of the sensor member, as well as facilitating increased flexibility of the sensor member to move together with the body and reduce displacement. The enlarged distal end portion further provides a counterforce against pulling out, so that movement of the patient may translate to less axial movement of the sensor member and more flexion by the sensor member, in order to dampen the effects by movement around the implantation site on the positioning of the sensor member under the skin, reduce or prevent micromotions of the sensor member relative to the surrounding skin/tissue, and further improve retention of the sensor member in the skin.

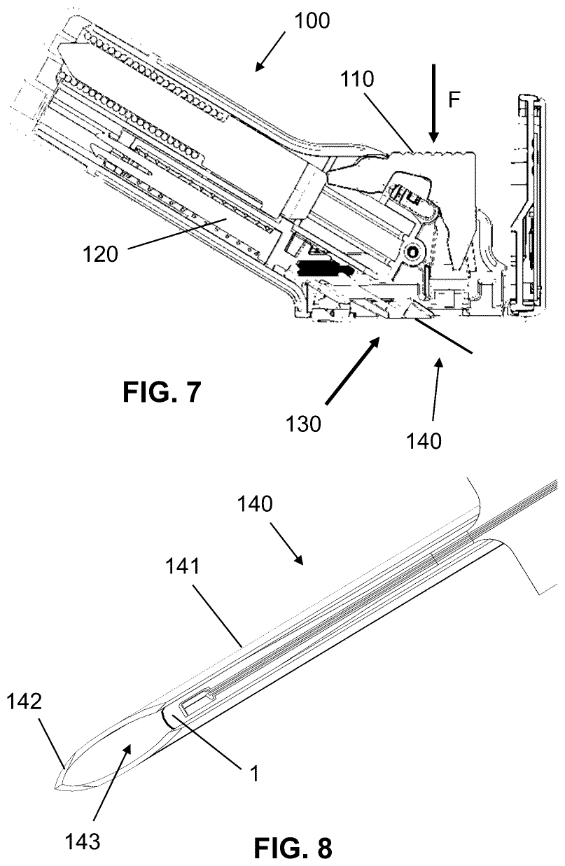

FIG. 7 shows a cross-sectional view from a side of an applicator according to an embodiment of the invention configured to implant a sensor member of an analyte monitor according to an embodiment of the invention. It is noted that the applicator shown in FIG. 7 is only an example, and that other applicators with different parts and designs have also been contemplated and should be covered within the spirit and scope of the invention, so long as the parts and/or procedures described below or their respective equivalents are included. It should also be noted that other parts of the applicator that are not vital to the inventive concepts of the invention may not be described in great detail herein, and that other designs can be incorporated to also accomplish the functional aspects of applicators according to embodiments of the invention, so long as the inventive concepts described herein are included in the applicator.

The applicator 100 according to the embodiment shown in FIG. 7 will generally include a main body with various components housed in the main body and attached thereto. Most importantly, the applicator will include a button, trigger, or other actuator 110 activating the applicator, a plunger or other advancement mechanism 120 for deploying a delivery needle 140 or other carrier for the sensor member 1, a lower surface 130 configured to face the patient's skin which may include features for holding a base of a monitoring system to be applied to the patient's skin, and the delivery needle 140 that is configured to implant part of the sensor member 1 under the patient's skin. Here, the delivery needle 140 may be attached to the plunger 120 so that advancement of the plunger 120 will result in advancement of the delivery needle 140. The plunger 120 may be latched or otherwise held at an initial or storage position, with the delivery needle retracted relative to the lower surface 130, and the actuator 110 may be depressed or otherwise actuated to release the latching mechanism and to advance the plunger 120 and delivery needle 140 past the lower surface 130 and into and under the patient's skin.

In operation, the lower surface 130 of the applicator will include one or more features to facilitate holding of a base of a monitor thereto, for example, a releasable latch. The base of the monitor, and in some cases part of the applicator, will include an adhesive to facilitate placement of the base of the monitor at a desired location on the patient's skin. The actuator 110 is then depressed to release and advance the plunger 120, which pushes the delivery needle 140 into the patient's skin, typically with an entry site within the perimeter of the adhered base of the monitor. The delivery needle 140 will advance a sensor member 1 housed therein into the skin, and then the sensor member 1 will be held in place under the skin, while the delivery needle 140 is retracted and separated from the base and sensor member assembly, and then the entire applicator 100 can be removed from the application site.

In some embodiments, the latch or other releasable mechanism on the lower surface 130 of the applicator will release the base of the monitor after the sensor member 1 has been delivered under the patient's skin, to facilitate separation and removal of the applicator after the base and sensor member have been properly applied to the patient. In other embodiments, the latch or other releasable mechanism on the lower surface 130 of the applicator may release the base of the monitor earlier, for example, prior to the tip 142 of the delivery needle 140 reaching the implantation site, or more generally before the delivery needle 140 punctures the skin of the patient and before the sensor member 1 is implanted into the patient. This latter arrangement, that is, an earlier release of the base of the monitor from the applicator, may be beneficial in various different ways. For example, current applicators often run into issues where patients have trouble disengaging the applicators from a fully implanted cradle or monitor due to insufficient detachment mechanisms in the current designs. Early release of the cradle reduces or eliminates such situations from arising, and the early release on the connection should further allow for some wiggle room during deployment, which may further increase comfort for the patient.

FIG. 8 shows an enlarged perspective view of the delivery needle or other sharp 140 of the applicator 100 according to an embodiment of the invention. The delivery needle 140 may include a main body 141, a sharp or other tip 142 at a distal end of the main body 141 that is suitable for piercing a surface of the patient's skin prior to advancement of the delivery needle 140 into the skin, and a hollow interior 143 that is sized and shaped to facilitate positioning of at least the distal end of the sensor member 1 therein, as shown in FIG. 8. The main body 141 of the needle may further include a longitudinal slit to provide lateral access into the hollow interior 143, which may facilitate insertion into and removal of the sensor member 1 from the delivery needle 140.

As illustrated, a width of the longitudinal slit may be, for example, wider than the thinner width of the main body of the sensor member 1, but narrower than the wider distal end of the sensor member 1. In this manner, it may be easier, for example, to facilitate lateral insertion and removal of the sensor member 1 to and from the delivery needle 140. Specifically, sensor member 1 may be inserted into the delivery needle 140, for example, when the larger distal end of the sensor member is more distally positioned than as illustrated, so as to be more easily inserted into the wider opening near the distal tip 142 of the delivery needle 140, while the narrower portion of the sensor member 1 can be laterally inserted through the longitudinal slit into the delivery needle 140 to allow for easier assembly. The sensor member 1 can then be pulled proximally into the hollow interior 143, so that the wider distal end of the sensor member 1 is pulled into the portion of the delivery needle 140 with the longitudinal slit, e.g., as illustrated in FIG. 8. In this configuration, the wider distal end of the sensor member 1 will prevent lateral removal of the sensor member through the longitudinal slit of the delivery needle 140. Furthermore, the wider distal end portion of the sensor member 1 may in some embodiments be oversized, for example, such that the wider distal end portion is slightly folded, bent, or otherwise deformed, to provide a slight interference fit with the delivery needle, to further increase a hold and retention of the sensor member 1 in the delivery needle 140 during storage and/or more generally prior to delivery/implantation. Thereafter, upon deployment, the sensor member 1 will be delivered together with the delivery needle 140 into the skin to a desired depth, and then the delivery needle 140 will begin retracting, i.e., moving proximally. Adhesion of the sensor member 1, for example, to the base or to another portion of the monitor or the patient's skin, will result in the sensor member 1 being held in place at the desired position under the skin. Meanwhile, retraction of the delivery needle 140, for example, up and to the right as illustrated, while the sensor member 1 stays stationary, will result in the wider distal end of the sensor member 1 moving distally relative to the delivery needle 140 until the wider distal end again reaches the distal tip portion 142 of the delivery needle with the widened opening, thereby facilitating easier release of the wider distal end of the sensor member 1 from the hollow interior 143 of the delivery needle 140. Due to the undersize of the width of the main body of the sensor member 1 compared to the width of the longitudinal slit of the delivery needle 140, the narrower portions of the sensor member 1 can separate from the delivery needle 140 either through the distal opening or laterally through the longitudinal slit, or partially both, depending on the arrangement of the parts and particular retraction of the delivery needle 140 during application/implantation of the sensor member 1. When the sensor member 1 successfully separates from the delivery needle 140, the delivery needle 140 can then be retracted to a safe position within the applicator while the sensor member 1 stays at a desired position under the patient's skin, and the delivery needle 140 and the rest of the applicator 100 can then safely be separated from the base and sensor and removed from the patient's skin.

Further improvements to the application of the monitor can also be incorporated according to embodiments of the invention. First, as noted previously, the base or frame of the monitor to be applied by the applicator may include an adhesive or other attaching means. In some embodiments, adhesion of the base is via an adhesive patch. The adhesive patch may be sized and shaped to constrain the skin around the entry site for the delivery needle 140. In other embodiments, the base of the monitor may be adhered to the skin via a first adhesive, and the lower side 130 of the applicator 100 itself may include a portion configured to directly engage the patient's skin within the perimeter of the base of the monitor, where the engagement portion may also include a separate adhesive patch or other adhesive to constrain the area of the skin around the entry site for the delivery needle 140. As previously discussed, according to embodiments of the invention, the applicator 100 will facilitate entry of the delivery needle 140 into the patient's skin at a relatively sharp angle, e.g., 30° or more broadly somewhere between 20° and 40°, relative to the surface of the patient's skin. Typically, entry of the delivery needle 140 will follow a linear trajectory, but other embodiments are also conceivable, for example, circular or rotational entry via a half-moon needle. Whether on the bottom surface of the base of the monitor or on a separate portion of the applicator that engages the skin, the adhesive patch for facilitating needle insertion may have a small hole through it for the delivery needle 140 to extend through to pierce the skin. Therefore, since the pre-adhered patch maintains a static position of the skin and keeps the skin relatively taut at the insertion site or entry site during deployment of the delivery needle 140, the skin around the insertion site is thoroughly constrained when the delivery needle 140 is advanced into the patient's skin. This allows for a more accurate positioning of the delivery needle 140 during insertion, more accurate entry at a desired angle, and reduced damage or trauma to the surrounding tissue and less discomfort for the patient.

A rigid frame, either via the base or via the applicator, will support the adhesive patch during application to ensure that the skin is constrained as close to the insertion site as possible. For example, additional tension may be applied via the patient or assistant when pushing down on the applicator, where the downward force applied on the applicator can work together with the positioning of the adhesive patch to further enhance the constraint on the skin around the needle insertion site. While in general, more traditional applicators and monitors have included adhesive patches to keep their respective devices adhered to the patient, the adhesive patch in more traditional applications includes a contact ring that is farther away from the insertion site. More specifically, for some competitors, when a monitor is adhered to the patient, the associated applicator may form a contact ring (e.g., contact formed when the patient pushes down on the applicator) that is closer to the outer perimeter of the monitor or around the edge of the applicator, which may in some cases be as much as 1″ to 3″ away from the insertion site. This increased distance of the contact ring from the actual insertion site reduces the constraint or tension applied onto the surface of the skin close to the insertion site, so that the skin around the insertion site may be looser during insertion of the delivery needle 140, which may result for example, in reduced accuracy as to the positioning and angle of insertion of the delivery needle 140, as well as increased trauma at the insertion site and discomfort to the patient.

In contrast, the applicator and base combination according to embodiments of the invention will include at least one feature, whether on the base or on the applicator, which will facilitate direct contact with the skin close to the insertion site of the delivery needle 140. In preferred embodiments, the direct constraint on the skin surrounding the insertion site that is caused by the assembly may be less than a centimeter away from the insertion site (or at most by less than one inch as an example), to maximize tension and constraint on the skin at and directly surrounding the insertion site. Again, this may be accomplished, for example, either via a contact ring formed on the base close to the insertion site that is pushed directly against the skin during deployment, or on a separate portion of the applicator that pushes directly against the skin inside of the perimeter of the base during deployment.

Furthermore, as seen in FIG. 7, although deployment of the delivery needle 140 is at an angle to the surface of the skin, the actuator 110 is actually depressed or otherwise actuated directly downwardly, or in other words, substantially perpendicularly to the surface of the skin, i.e., substantially in the direction of the arrow of applied force F illustrated in FIG. 8. In addition, the position of the actuator 110 is also purposefully located directly above the insertion site, so that pressing down on the actuator 110 will also cause additional pressure to be applied directly onto the insertion site. With the trigger force of the actuator 110 being oriented and applied normal to the surface of the skin as well as directly above the position of the insertion site, the pre-adhered patch will have a pressure applied by the patient or other person helping with the application that is oriented directly downward onto the surface of the skin at the insertion site, further enhancing any pressure applied at the contact ring around the insertion site. Such an arrangement allows pressure to be applied at the insertion site that will consistently meet a minimum compression against the skin that is needed to ensure sufficient tensioning or constraint of the skin at the insertion site according to embodiments of the invention. In some embodiments, the force F necessary to release the actuator 110 and activate the applicator is set or configured to be greater than a desired pressure to be applied at the insertion site, to ensure sufficient pressure is applied thereto. In other embodiments, the release mechanism for the actuator 110 may have a controlled stiffness and location, such that a consistent force is configured to be applied at the deployment site when the actuator 110 is actuated. This arrangement is different from other more traditional applicators that deploy sensors at an angle or vector corresponding to the delivery angle, with actuation mechanisms that are arranged parallel to the movement of the delivery needle, and thus not perpendicular to the insertion site, thereby both reducing and angling a pressure that is applied by the applicator at the insertion site, and thus reducing the amount of pressure applied as well as the tension or constraint on the skin.

According to embodiments of the invention, the system and method is optimized for introducing a sensor member 1 to a depth of about 1.5 mm to about 3 mm deep into the skin, and more preferably less than 2.5 mm deep to ensure positioning of sensing elements in the dermal layer. By first adhering an adhesive patch to the skin, and constraining it more locally to the insertion site (e.g., within 1 cm around the insertion site), the skin around the insertion site can be ensured to be sufficiently taut and constrained at the time of insertion of the sensor member 1. A user operated release mechanism then actuates the delivery needle 140 to advance and pierce the skin to deliver the sensor member 1, and then retract while leaving the sensor member 1 behind at the target depth, while the base or the carrier of the analyte monitor is attached to the adhesive patch frame. Positioning and direction of the actuation module will further help facilitate sufficient pressure being applied at the insertion site, so that the skin is sufficiently constrained and prepared for needle insertion.

Other embodiments may also be envisioned without departing from the spirit or scope of the invention. For example, in some embodiments, rather than the adhesive patch being attached to a base, frame, or carrier of the analyte monitor, the adhesive patch may instead be intended to attach a transmitter of the analyte monitor, or in other embodiments, may be intended to attach the entire analyte monitor, particular in embodiments where the analyte monitor is arranged in a single integral main body. Various other modifications can also be incorporated while retaining the inventive concepts of the present application.

In addition to the embodiments that have already been described above, it is also possible to combine embodiments, e.g., different features from the various described embodiments, to provide even more different variations of sensor members, associated analyte monitors, and/or applicators, without departing from the spirit or scope of the invention. In addition, the inventions should not be limited to the structures and/or shapes described in the embodiments above.

While the subject matter of the present disclosure has been described in connection with certain embodiments, it is to be understood that the subject matter of the present disclosure is not limited to the disclosed embodiments, but, on the contrary, the present disclosure is intended to cover various modifications and equivalent arrangements included within the spirit and scope of the appended claims, and equivalents thereof.

What is claimed is:

1. An applicator for applying an analyte monitor to a patient's skin, the applicator comprising:
a surface configured to hold at least a base of the analyte monitor for positioning the base against the patient's skin and adhering the base to the patient's skin;
a delivery needle configured to advance axially past the surface and the base when the base is held to the applicator to deliver a portion of a sensor member of the analyte monitor into the patient's skin at an insertion site located laterally within a perimeter of the base; and
an actuator configured to be actuated to initiate advancement of the delivery needle to the insertion site and into the patient's skin;
wherein when the surface of the applicator is held against the patient's skin to adhere the base to the patient's skin, at least part of the actuator is positioned directly above the insertion site of the delivery needle relative to an axis that is normal to a surface of the patient's skin, and the actuator is configured to be actuated in a direction parallel to the axis that is normal to the surface of the patient's skin.

2. The applicator of claim 1, wherein the surface of the applicator is further configured to hold at least a transmitter of the analyte monitor.

3. The applicator of claim 1, wherein the delivery needle is configured to advance linearly past the surface and the base when the actuator is actuated.

4. The applicator of claim 3, wherein the delivery needle is configured to advance linearly at an acute angle relative to the surface of the patient's skin.

5. The applicator of claim 1, wherein the delivery needle is configured to advance along a curved trajectory past the surface and the base when the actuator is actuated.

6. The applicator of claim 1, wherein after the portion of the sensor member is delivered into the patient's skin at the insertion site, the delivery needle is configured to be retracted back above the surface and the base and into the applicator while the portion of the sensor member remains in the patient's skin.

7. The applicator of claim 1, wherein the actuator comprises a button or trigger.

8. The applicator of claim 1, wherein a minimum force needed to actuate the actuator corresponds to a desired force to be applied by the actuator to the patient's skin at the insertion site to constrain a region of the patient's skin around the insertion site.

9. An applicator for applying an analyte monitor to a patient's skin, the applicator comprising:
a surface configured to hold at least a base of the analyte monitor for positioning the base against the patient's skin and adhering the base to the patient's skin;
a delivery needle configured to advance axially past the surface and the base when the base is held to the applicator to deliver a portion of a sensor member of the analyte monitor into the patient's skin at an insertion site located laterally within a perimeter of the base; and
an actuator configured to be actuated to initiate advancement of the delivery needle to the insertion site and into the patient's skin;
wherein when the surface of the applicator is held against the patient's skin to adhere the base to the patient's skin, the delivery needle is configured to be advanced along an axis that is arranged at an acute angle relative to a surface of the patient's skin to deliver the sensor member under the surface of the patient's skin at the acute angle, while the actuator is configured to be actuated in a direction perpendicular to the surface of the patient's skin, such that respective axes of movement of the delivery needle and the actuator are angled relative to one another.

10. The applicator of claim 9, wherein the surface of the applicator is further configured to hold at least a transmitter of the analyte monitor.

11. The applicator of claim 9, wherein after the portion of the sensor member is delivered into the patient's skin at the insertion site, the delivery needle is configured to be retracted back above the surface and the base and into the applicator while the portion of the sensor member remains in the patient's skin.

12. The applicator of claim 9, wherein the actuator comprises a button or trigger.

13. The applicator of claim 9, wherein the acute angle is between 20° and 40°.

14. The applicator of claim 13, wherein the acute angle is approximately 30°.

15. A system comprising an analyte monitor and an applicator for applying the analyte monitor to a patient's skin, the applicator comprising:
a surface configured to hold at least a base of the analyte monitor for positioning the base against the patient's skin;
a delivery needle configured to advance axially past the surface and the base when the base is held to the applicator to deliver a portion of a sensor member of the analyte monitor into the patient's skin at an insertion site located laterally within a perimeter of the base; and
an actuator configured to be actuated to initiate advancement of the delivery needle to the insertion site and into the patient's skin;
wherein when the surface of the applicator is held against the patient's skin, an adhesive patch is configured to adhere the base to the patient's skin, the adhesive patch further configured to constrain a region of the patient's skin surrounding the insertion site, and wherein actuation of the actuator is configured to apply direct pressure against a portion of the adhesive patch that surrounds the insertion site and that is closer radially to the insertion site than to an outer perimeter of the base to increase tension of the patient's skin that surrounds the insertion site during advancement of the delivery needle.

16. The system of claim 15, wherein the surface of the applicator is further configured to hold at least a transmitter of the analyte monitor.

17. The system of claim 15, wherein after the portion of the sensor member is delivered into the patient's skin at the insertion site, the delivery needle is configured to be retracted back above the surface and the base and into the applicator while the portion of the sensor member remains in the patient's skin.

18. The system of claim 15, wherein the sensor member is configured to be inserted to a maximum depth of 1.5 mm to 3 mm deep in the patient's skin.

19. The system of claim 18, wherein the sensor member is configured to be inserted to a maximum depth of less than 2.5 mm deep in the patient's skin, so as to position sensing regions of the sensor member in the dermis of the patient's skin.

20. The system of claim 15, wherein when the surface of the applicator is held against the patient's skin, a distance between the insertion site and the portion of the adhesive patch that surrounds the insertion site where the direct pressure is applied by the actuator is less than 1 cm.
